(19) United States
(12) Patent Application Publication
(10) Pub. No.: US 2006/0178426 A1
(43) Pub. Date: Aug. 10, 2006

(54) Kappa agonists, especialy for the treatment and/or prophylaxis of irritable bowel syndrome

(76) Inventors: Wolfgang Stahle, Ingelheim (DE); Rudolf Gottschlich, Reinheim (DE); Jurgen Harting, Darmstadt (DE); Christoph Seyfried, Seeheim-Jugenheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/563,975

(22) PCT Filed: Jun. 18, 2004
(86) PCT No.: PCT/EP04/06630

§ 371 Date: Jan. 10, 2006

(30) Foreign Application Priority Data
Jul. 11, 2003 (DE) 103 31 723.6

(51) Int. Cl. A61K 31/4015; C07D 207/46

(52) U.S. Cl. 514/424; 548/542

(57) ABSTRACT

Compound of the formula (I), in which A, R¹, R², R³, X, Y, m and n have the meaning indicated, are suitable as for the treatment of irritable bowel syndrome.

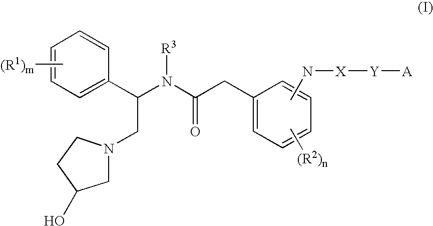

The invention relates to compounds of the formula I 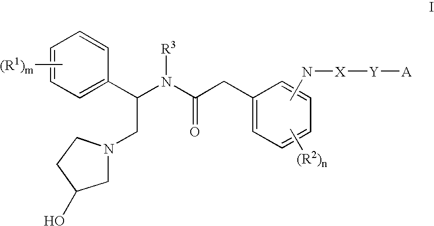
in which
A denotes a mono- or bicyclic aromatic or non-aromatic carba- or heterocyclic ring system which is unsubstituted or mono- or polysubstituted by R¹,
R¹ denotes H, Hal, NO₂, NHR, NRR, OR, CO—R, SO₃R, SO₂R, SR, CF₃, OCF₃, SCF₃, C₁-C₈ alkyl, C₃-C₁₄ cycloalkyl,
R₂ denotes H, Hal, NO₂, NHR, NRR, OR, CO—R, SO₃R, SO₂R, SR, CF₃, OCF₃, SCF₃, C₁-C₈ alkyl, C₃-C₁₄ cycloalkyl, R₃ denotes C₁-C₈ alkyl,
x denotes CO, CS, SO₂,
Y denotes a single bond, O, NH, CH₂
R denotes H or a C₁-C₈ alkyl, C₃-C₁₄ cycloalkyl, C₆-C₁₀ aryl or C₇-C₁₄ aralkyl group, which may be mono- or polysubstituted by R₅ and whose alkyl-C chain may be interrupted by —O—,
Hal denotes F, Cl, Br, or I
m denotes 0,1, 2, 3 or 4
and
n denotes 0, 1, 2 or 3,
and/or one of their physiologically acceptable salts and/or one of their glycosylated derivatives.

Compounds having a similar structural formula and suitable processes for their preparation are described in DE-A 198 49 650, DE 40 34 785 and DE 42 15 213. The use of similar compounds for the treatment of inflammatory intestinal diseases is disclosed in EP 0 752 246. It was an object of the invention to provide pharmaceutically effective compounds which can be employed and are effective, in particular, in the treatment and/or prophylaxis of irritable bowel syndrome (IBS or colon irritable) which simultaneously ameliorate the pain associated with this disease and cure the disease.

At the same time, it was an object of the invention to provide pharmaceutically effective compounds which have no effects on normal intestinal peristalsis, but contribute to the curing of irritable bowel syndrome. IBS is the commonest cause of abdominal pain syndromes.

Preferred compounds of the formula I are kappa agonists, in particular peripherally acting kappa agonists, and are therefore suitable for the treatment of diseases which, as is known, can be influenced by kappa agonists, such as, for example, pruritus (U.S. Pat. No. 6,004,964). The compounds are likewise suitable as analgesics.

It has now been found that compounds of the formula I 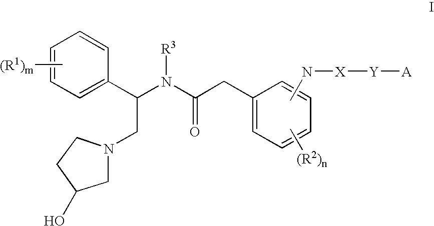
in which A, R¹, R², R³, X, Y, m and n have the meanings indicated above and/or physiologically acceptable salts thereof and/or glycosylated derivatives thereof, are pharmaceutically active compounds which are particularly suitable as kappa agonists and active ingredients in medicaments for the treatment of irritable bowel syndrome. In particular, preference is given to compounds of the formula IA 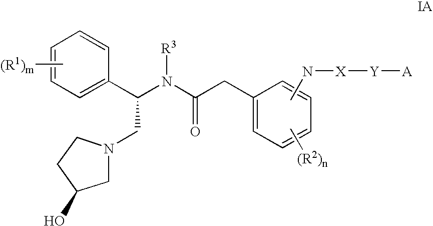
in which A, R¹, R², R³, X, Y, m and n have the meanings indicated above. Very particular preference is given to compounds of the formula I and IA
in which
A denotes phenyl, pyridyl, thienyl or cyclohexyl, each of which is unsubstituted or mono- or polysubstituted by R¹,
R¹ denotes H
R² denotes H or Hal.

Preference is also given to compounds of the formula I and IA in which
A denotes phenyl or naphthyl
and/or
x denotes CO or SO₂, in particular SO₂
and/or
Y denotes a single bond or NH.

Hal preferably denotes F, Cl or Br, in particular Cl.

Besides the compounds of the formula I, the invention thus relates to the use of the compounds of the formula I as medicaments for the treatment of diseases which can be influenced by kappa agonists, and in particular of irritable bowel syndrome. The present application also relates to compositions which comprise compounds of the formula I as constituent for the treatment and/or prophylaxis of irritable bowel syndrome.

Experiments have shown that the compounds according to the invention act on mice or rats in the “writhing test” (method cf. Siegmund et. al., Proc. SOC. Exp. Biol. 95, (1957), 729-731). The analgesic action as such can furthermore be demonstrated in the “tail-flick test” on mice or rats (method cf. &Amour and Smith, J. Pharmacol. Exp. Ther. 72, (1941), 74-79), furthermore in the “hot plate test” (cf. Schmauss and Yaksh, J. Pharmacol. Exp. Ther. 228, (1984), 1-12 and the literature cited therein). Particularly strong actions can be observed in rats in the model of carrageenin-induced hyperalgesia (cf. Bartoszyk and Wild, Neuroscience Letters 101 (1989) 95). The compounds exhibit no or an only slight tendency towards physical dependence here.

In addition, corresponding experiments carried out by common methods have shown pronounced antiinflammatory, diuretic, anticonvulsive, neuro-protective actions. The compounds exhibit high affinity with respect to the binding behaviour to kappa receptors.

In contrast to other compounds having a similar activity spectrum, compounds of the formula I are particularly suitable for use in pharmaceutical compositions for the treatment of irritable bowel syndrome since, besides the analgesic and antiinflammatory action, they are suitable for normalising impairments in the intestinal motor system caused by the disease.

In addition, it has proven particularly advantageous in the case of the compounds according to the invention that, owing to their structure, they are apparently unable to pass through the blood/brain barrier and therefore have no dependency potential.

The compounds of the formula 1, they are, in addition, distinguished by the fact that, owing to their pharmacokinetic properties, such as, for example, a logD value <−1.5 or a very low solubility of less than 0.01 mol/l, they can only be absorbed to an extremely low proportion or not at all. They are therefore predestined for local use in the intestine.

In addition, no effects have hitherto been found which would in any way restrict the use of the advantageous effects for the claimed indications.

The compounds of the general formula I and physiologically acceptable salts thereof can therefore be used for the preparation of pharmaceutical preparations by bringing them into the suitable dosage form together with at least one excipient or adjuvant and, if desired, with one or more further active ingredients.

The invention therefore also relates to a pharmaceutical composition, characterised by a content of at least one compound of the formula I and/or one of its physiologically acceptable salts for the treatment of irritable bowel syndrome.

The compositions obtained in this way can be employed as medicaments in human or veterinary medicine. Suitable excipient substances are organic or inorganic substances which are suitable for enteral (for example oral or rectal) or parenteral administration and do not react with the novel compounds, for example water, vegetable oils, benzyl alcohols, polyethyene glycols, glycerol triacetate and other fatty acid glycerides, gelatine, soya lecithin, carbohydrates, such as lactose or starch, magnesium stearate, talc or cellulose.

Suitable for oral administration are, in particular, tablets, dragees, capsules, syrups, juices or drops. Of particular interest are film-coated tablets and capsules having gastric juice-resistant coatings or capsule shells. Suitable for rectal administration are suppositories, suitable for parenteral administration are solutions, preferably oily or aqueous solutions, furthermore suspensions, emulsions or implants.

The active ingredients claimed in accordance with the invention may also be lyophilised and the resultant lyophilisates used, for example, for the preparation of injection preparations.

The compositions indicated may be sterilised and/or comprise adjuvants, such as preservatives, stabilisers and/or wetting agents, emulsifiers, salts for modifying the osmotic pressure, buffer substances, dyes and/or aroma substances. If desired, they may also comprise one or more further active ingredients, for example one or more vitamins, diuretics, antiphlogistics.

The compounds of the formula I according to the invention are generally administered analogously to other known preparations which are commercially available for the claimed indications, preferably in doses between about 1 mg and 50 mg, in particular between 5 and 30 mg, per dosage unit. The daily dose is preferably between about 0.02 and 20 mg/kg, in particular 0.2 and 0.4 mg/kg, of body weight.

However, the specific dose for each individual patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

Examples are given below which serve to illustrate the invention, but do not limit the invention to the examples given.

In the following examples, “conventional work-up” means: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation.

All temperatures below are indicated in ° C.

The following parameters were observed for analysis by HPLC MS:
Column: Chromolith SpeedROD, 50×4.6 mm² (Order No. 1.51450.0001) from Merck
Method: Eluent A: water+0.1% of TFA (trifluoroacetic acid) Eluent B: acetonitrile+0.08% of TFA
Gradient (linear): t=0 min, A:B=80:20, t=3 to t=3.5 min: A:B=0:100

Abbreviations:
M+H: Molar peak of the mass spectrum
MW: Molecular weight
RT: Retention time

### EXAMPLE 1

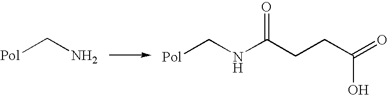

A mixture of 25.0 g of aminomethylated polystyrene resin (0.78 mmol/g), 20 mg of dimethylaminopyridine (DMAP) and 5.85 g of succinic anhydride in 200 ml of pyridine is stirred at room temperature (RT) for one day, giving after conventional work-up, the corresponding monoamide.

### EXAMPLE 2

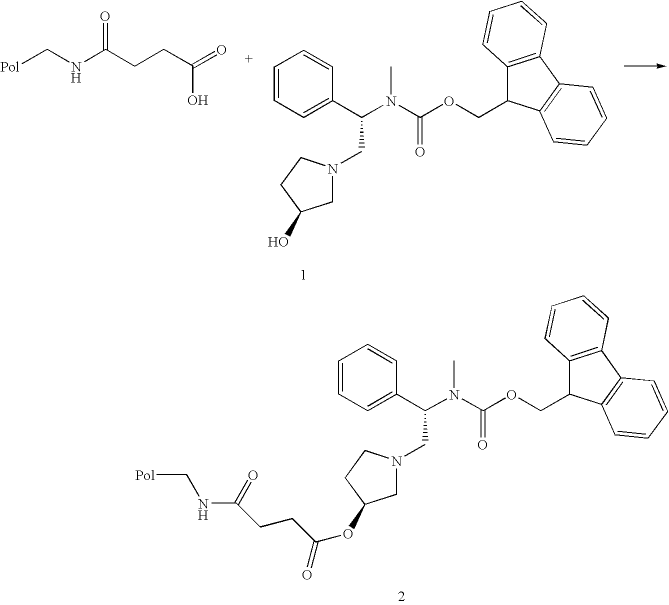

3.49 g of 1-(mesitylenesulfonyl)-3-nitro-1H-1,2,4-triazole (MSNT) and 4 ml of N-methylimidazole are added with stirring to a mixture of 7.91 g of the monoamide from Example 1 and 4.43 g of the compound 1 in 120 ml of methylene chloride. The mixture is stirred for 2 hours. Conventional work-up gives the ester 2 of the compound 1.

### EXAMPLE 3

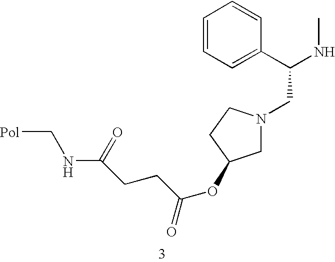

9.8 g of the ester 2 from Example 2 are stirred for 30 minutes in 30 ml of piperidine and 70 ml of dimethylformamide (DMF). Conventional work-up gives the compound 3.

### EXAMPLE 4

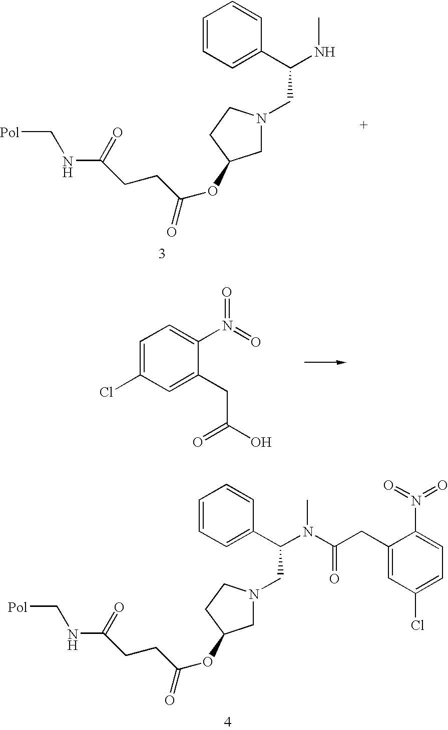

9.9 g of 2-nitro-5-chlorophenylacetic acid 14.8 g of 2-(1-H-benzotriazol-2yl)-1,1,3,3,-tetramethyluronium tetrafluoroborates (TBTU) and 11.9 g of diisopropyletylamine are added to a mixture of 7.644 mmol of the compound 3 in 130 ml of DMF. The reaction mixture is stirred at RT for 5 hours. Conventional work-up gives the amide 4.

### EXAMPLE 5

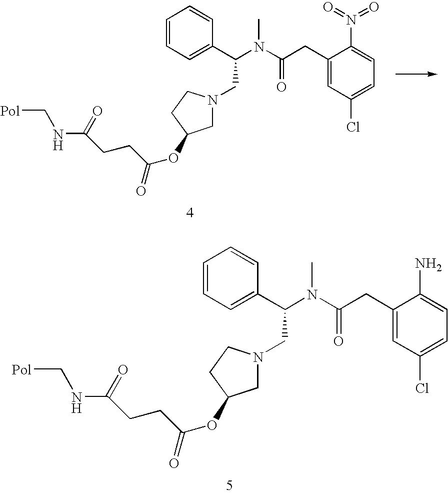

24.8 g of tin(II) chloride are added to a mixture of 9.4 g of the compound 4 in 130 ml of DMF, and the mixture is stirred at 50° C. for 6 hours. Conventional work-up gives the compound 5.

### EXAMPLE 6

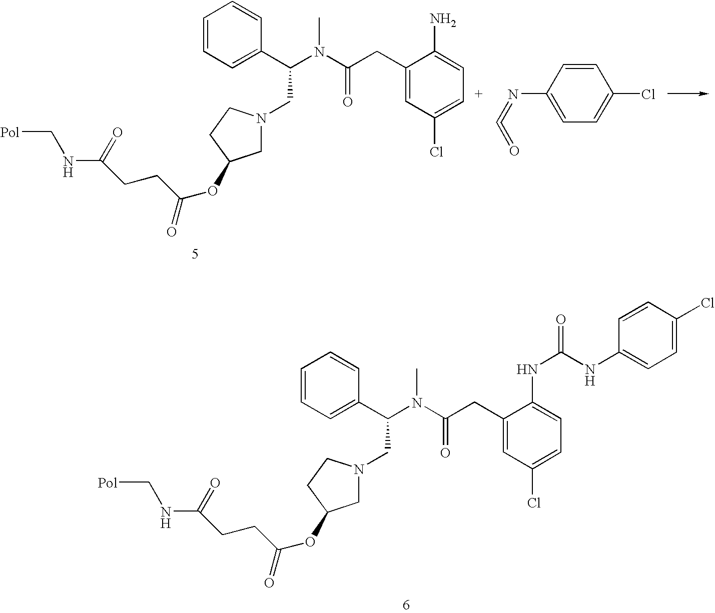

0.24 g of 4-chloropheyl isocyanate is added to a suspension of 0.2 g of the compound 5 in 2 ml of methylene chloride, and the mixture is stirred at RT for 18 hours. Conventional work-up gives the compound 6.

### EXAMPLE 7

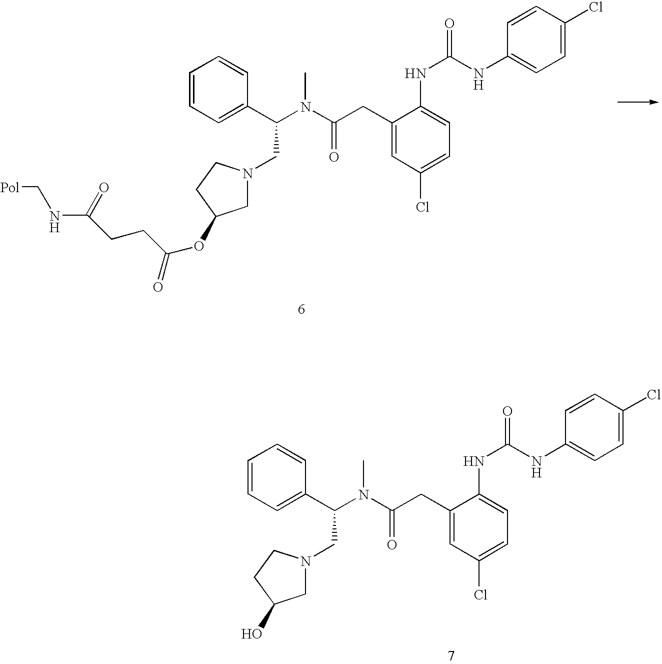

0.8 ml of 4N potassium hydroxide solution is added to a solution of 200 mg of the compound 6 in 4 ml of dioxane and 2 ml of methanol, and the mixture is stirred at RT for 5 hours. Conventional work-up gives the compound 7.

### EXAMPLE 8

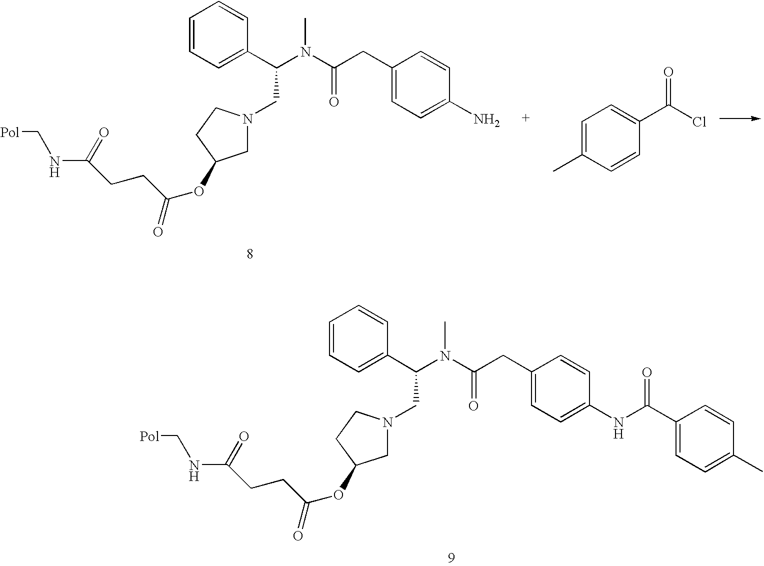

0.291 ml of 4-methylbenzoyl chloride and a spatula tip of DMAP are added to 0.15 g of the compound 8 in 1 ml of methylene chloride and 1 ml of pyridine. Conventional work-up gives the compound 9.

### EXAMPLE 9

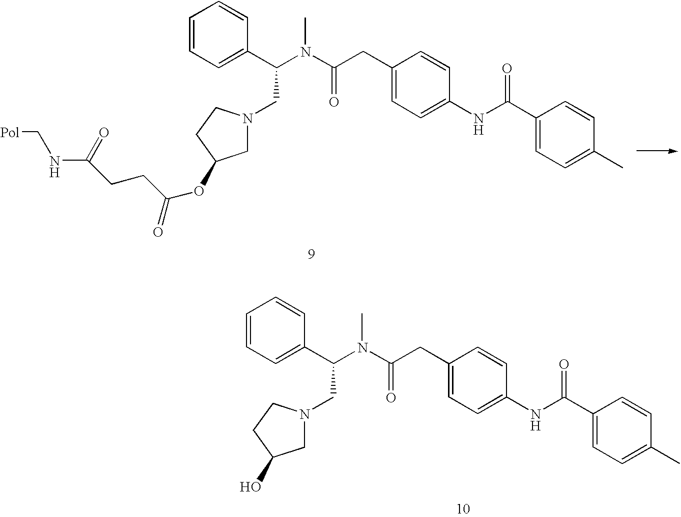

A mixture of 150 mg of the compound 9, 3.5 ml of dioxane, 1.8 ml of methanol and 0.7 ml of 4N potassium hydroxide solution is stirred at room temperature for 5 hours. Conventional work-up gives the compound 10.

### EXAMPLE 11

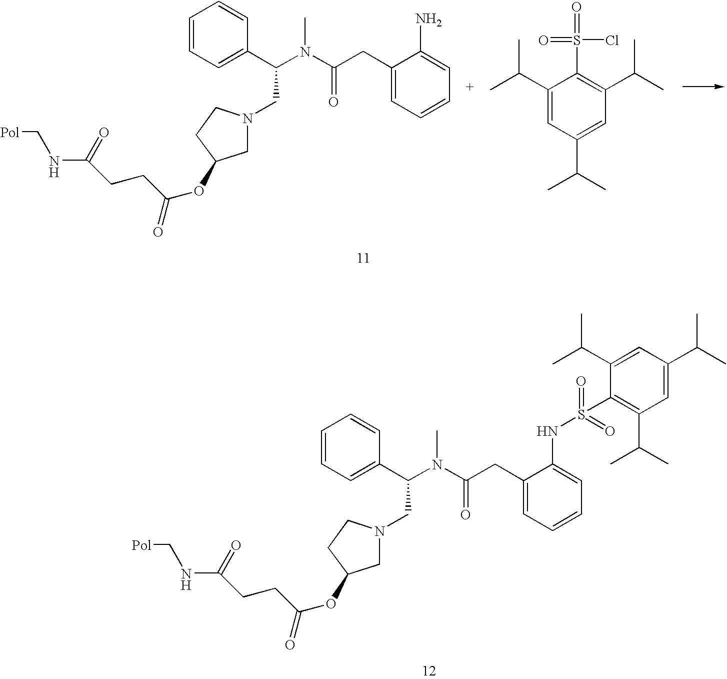

473 mg of 2,4,6-triisopropylbenzenesulfonyl chloride and a spatula tip of DMAP are added to 0.20 g of the compound 11 in 1 ml of methylene chloride and 1 ml of pyridine. The mixture is stirred for 3 hours. Conventional work-up gives the compound 12.

### EXAMPLE 12

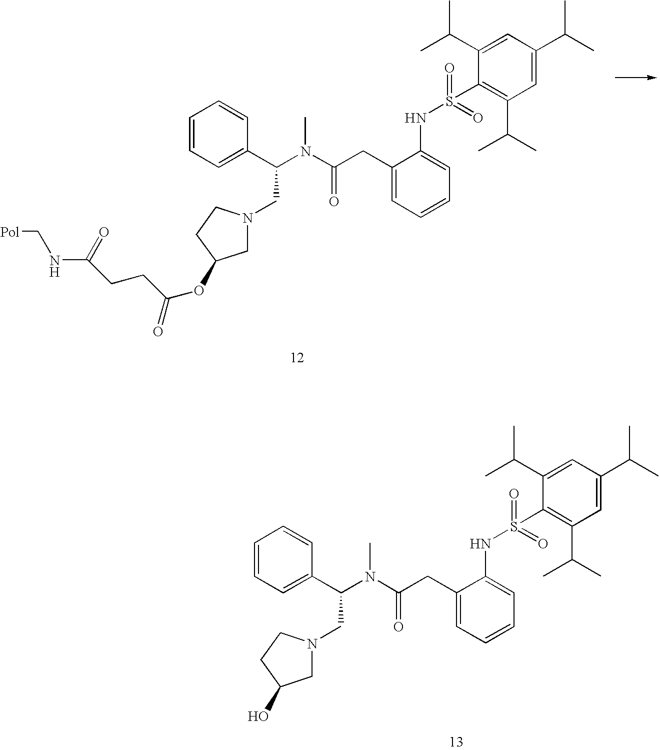

A mixture of 200 mg of the compound 12, 4 ml of dioxane, 2 ml of methanol and 0.8 ml of 4N potassium hydroxide solution is stirred at room temperature for 5 hours. Conventional work-up gives the compound 13.

The following compounds according to the invention are obtainable by using the corresponding precursors:

| Ref. No. | | RT (min) | M + H |
|---|---|---|---|
| 387714 | 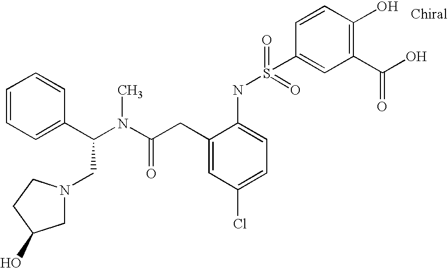 | 1.40 | 588 |
| 387721 | 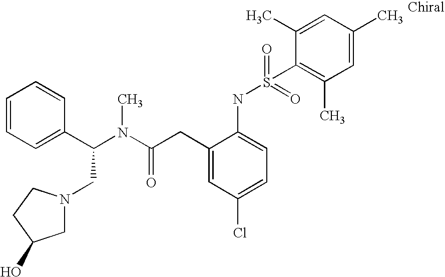 | 1.72 | 570 |
| 387731 | 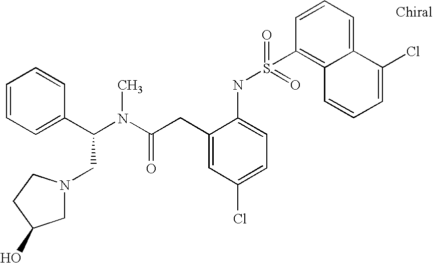 | 1.91 | 612 |
| 387732 | 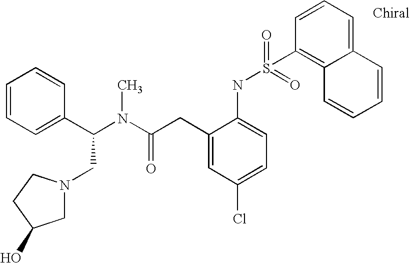 | 1.61 | 578 |
| 387733 | 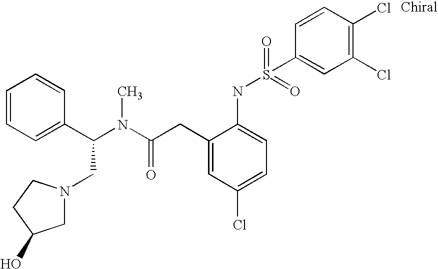 | 1.71 | 597 |
| 387734 | 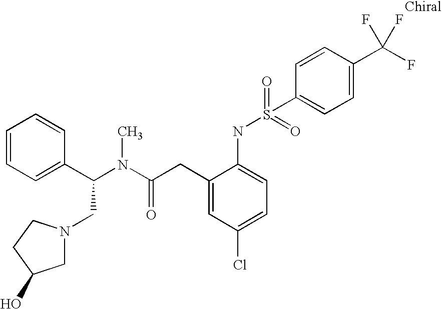 | 1.67 | 596 |
| 387735 | 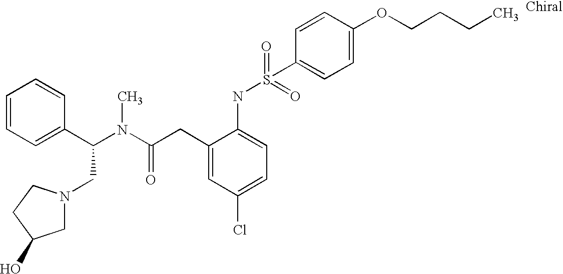 | 1.83 | 600 |
| 387736 | 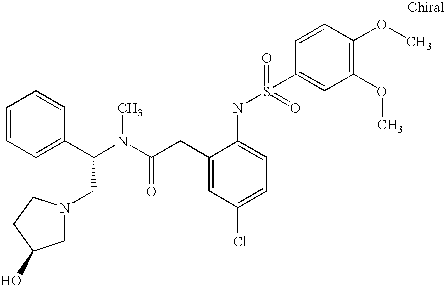 | 1.50 | 588 |
| 387737 | 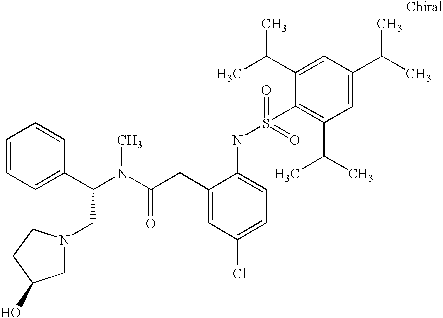 | 2.19 | 654 |
| 387738 | 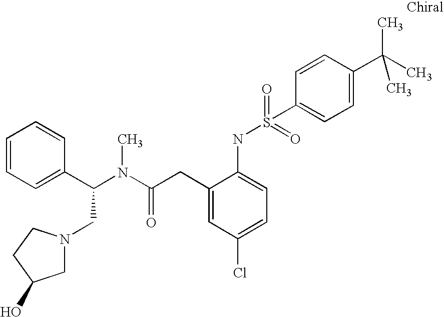 | 1.82 | 584 |
| 387739 | 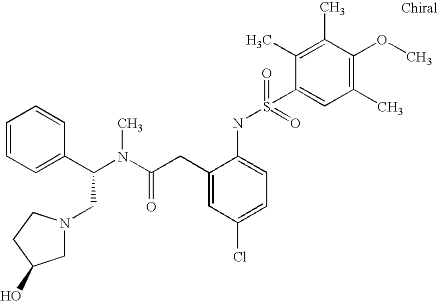 | 1.70 | 600 |
| 387743 | 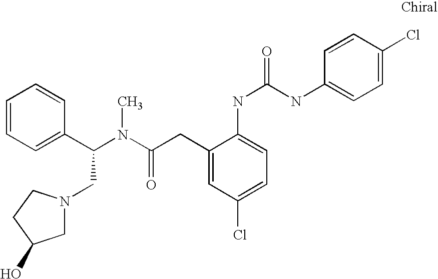 | 1.58 | 541 |
| 387744 | 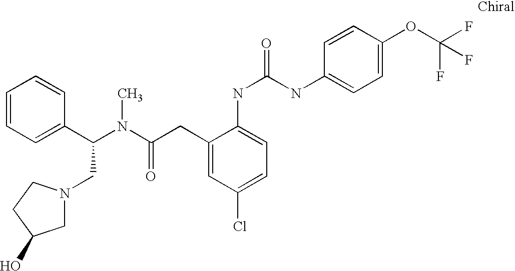 | 1.70 | 591 |
| 387745 | 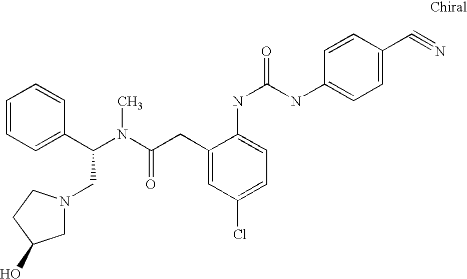 | 1.41 | 532 |
| 387748 | 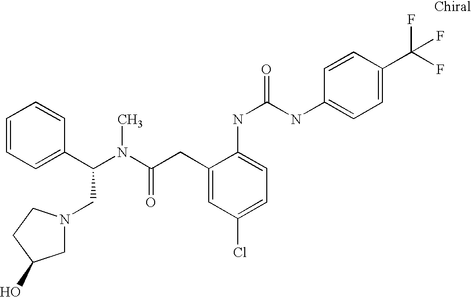 | 1.67 | 575 |
| 388748 | 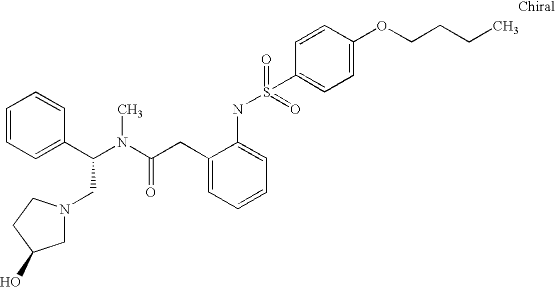 | 1.68 | 566 |
| 388750 | 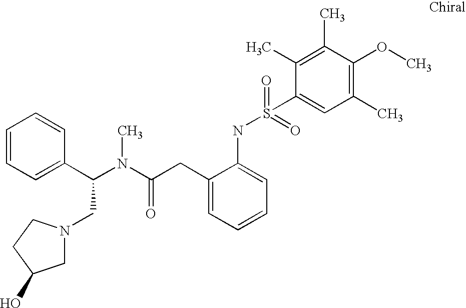 | 1.55 | 566 |
| 388753 | 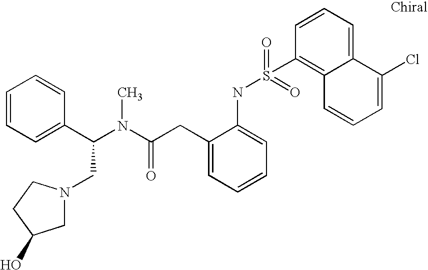 | 1.65 | 578 |
| 388756 | 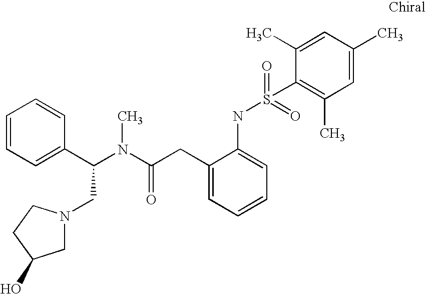 | 1.54 | 536 |
| 388758 | 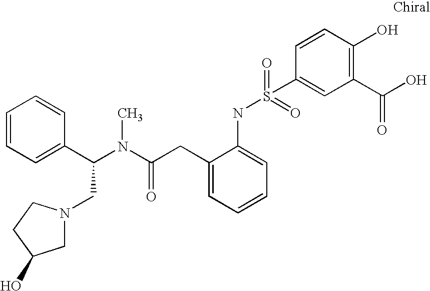 | 1.22 | 554 |
| 388808 | 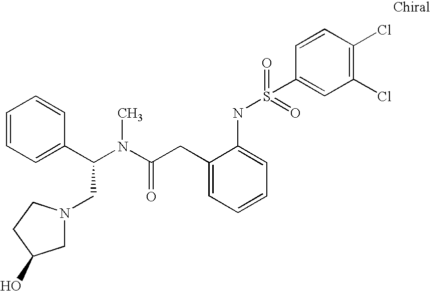 | 1.54 | 562 |
| 388809 | 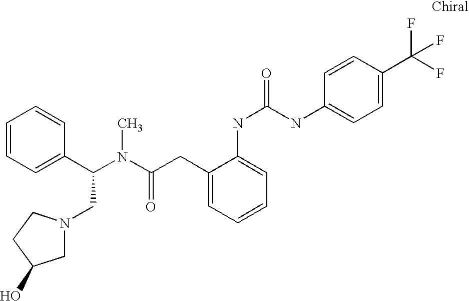 | 1.46 | 541 |
| 388810 | 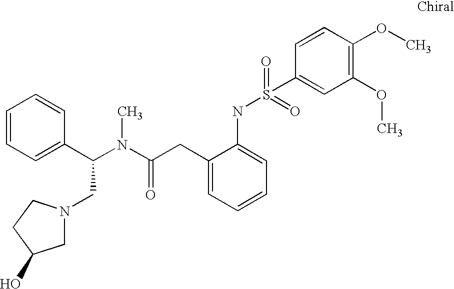 | 1.21 | 554 |
| 388811 | 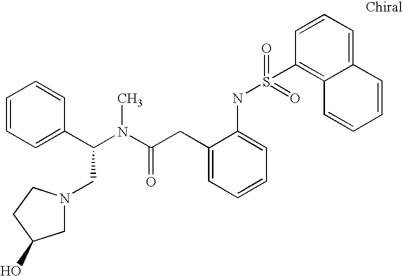 | 1.46 | 544 |
| 388813 | 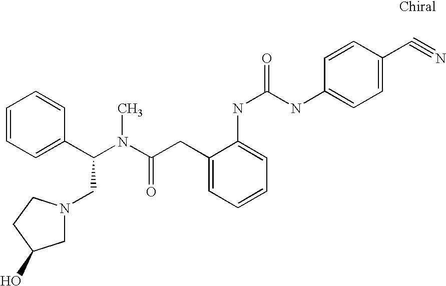 | 1.16 | 498 |
| 388814 | 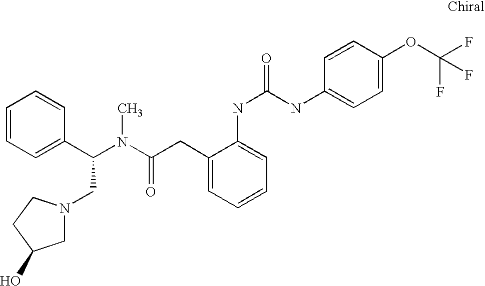 | 1.47 | 557 |
| 388815 | 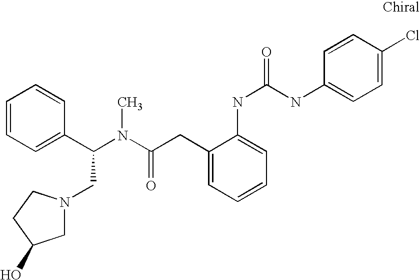 | 1.33 | 507 |
| 390485 | 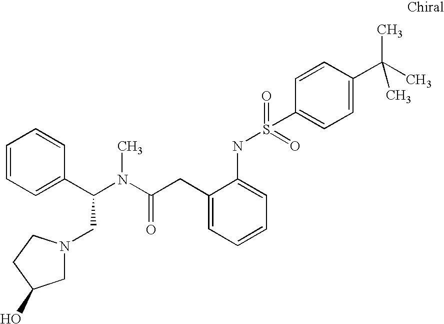 | 1.64 | 550 |
| 390486 | 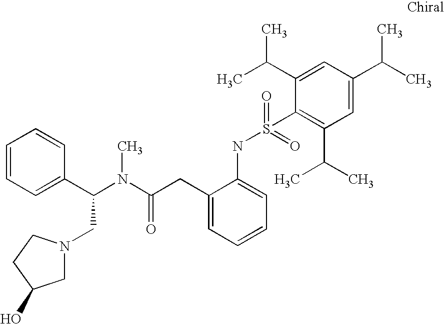 | 2.05 | 620 |
| 391182 | 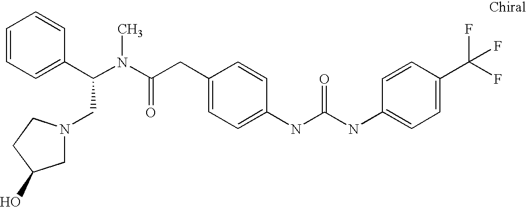 | 1.58 | 541 |
| 391183 | 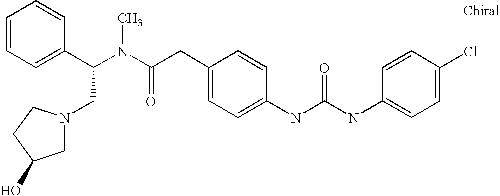 | 1.47 | 507 |
| 391185 | 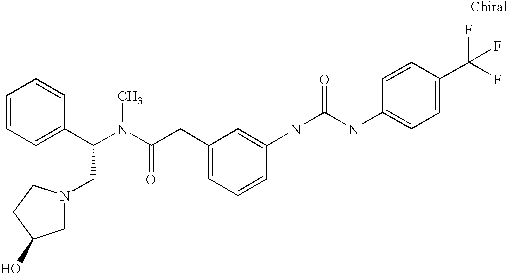 | 1.42 | 541 |
| 391186 | 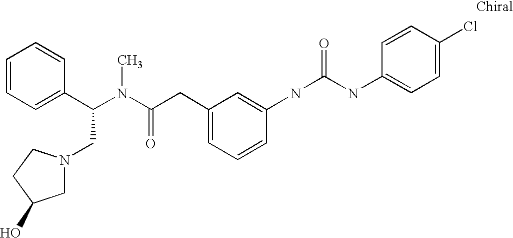 | 1.47 | 507 |
| 391193 | 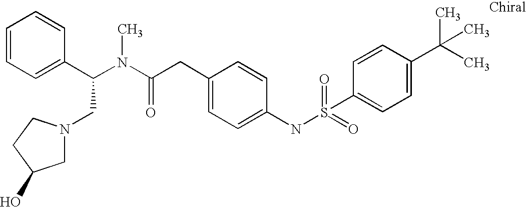 | 1.56 | 550 |
| 391194 | 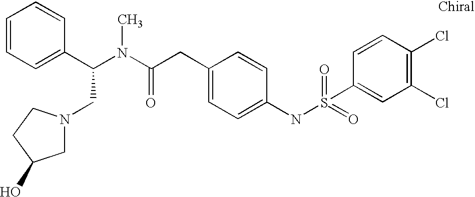 | 1.50 | 562 |
| 391195 | 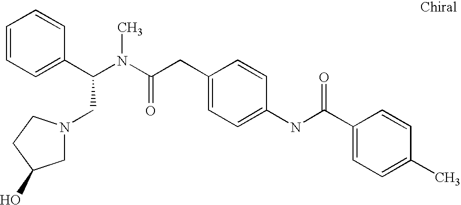 | 1.33 | 472 |
| 391196 | 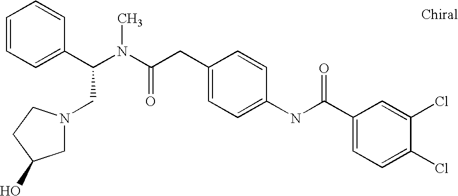 | 1.55 | 526 |
| 391203 | 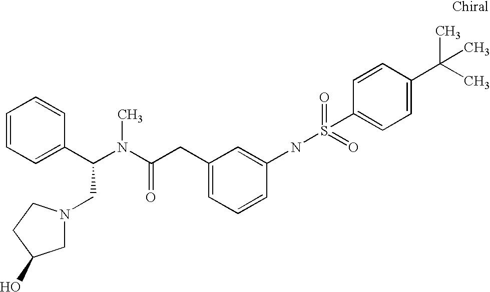 | 1.61 | 550 |
| 391204 | 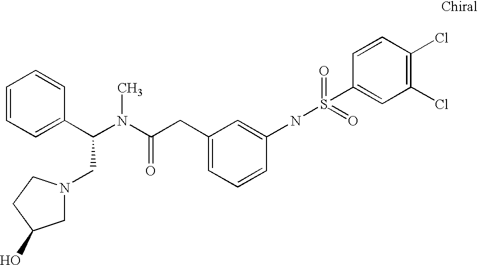 | 1.49 | 562 |
| 391205 | 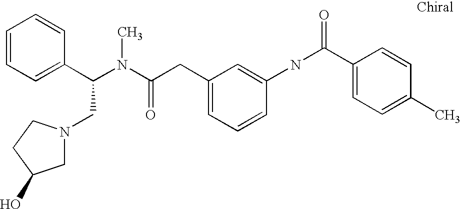 | 1.31 | 472 |
| 391207 | 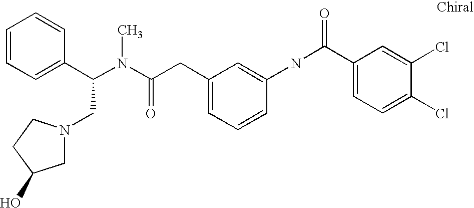 | 1.54 | 526 |

The pharmaceutical efficacy of the substances according to the invention in the treatment of irritable bowel syndrome can be investigated by the method described in European J. of Pharmacology 271 (1994) 245-251. The following examples relate to pharmaceutical compositions:

### EXAMPLE A

### Injection Vials

A solution of 100 g of an active ingredient of the formula I and 5 g of disodium hydrogenphosphate in 3 l of bidistilled water are adjusted to pH 6.5 using 2 N hydrochloric acid, sterile-filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE C

### Solution

A solution is prepared from 1 g of an active ingredient of the formula 1, 9.38 g of NaH₂PO₄, 2H₂O, 28.48 g of Na₂HPO₄, 12H₂O, and 0.1 g of benzalkonium chloride in 940 ml of bidistilled water. The pH is adjusted to 6.8, and the solution is made up to 1 l and sterilised by irradiation.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE F

### Dragees

Tablets are pressed analogously to Example E and subsequently coated in a conventional manner with a coating of sucrose, potato starch, talc, tragacanth and dye.

1. Compounds formula I 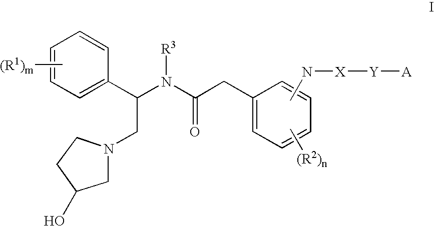
in which
A denotes a mono- or bicyclic aromatic or non-aromatic carba- or heterocyclic ring system which is unsubstituted or mono- or polysubstituted by R¹,
R¹ denotes H, Hal, NO₂, NHR, NRR, OR, CO—R, SO₃R, SO₂R, SR, CF₃, OCF₃, SCF₃, C₁-C₈ alkyl, C₃-C₁₄ cycloalkyl,
R₂ denotes H, Hal, NO₂, NHR, NRR, OR, CO—R, SO₃R, SO₂R, SR, CF₃, OCF₃, SCF₃, C₁-C₈ alkyl, C₃-C₁₄ cycloalkyl,
R₃ denotes C₁-C₈ alkyl,
X denotes CO, CS, SO₂,
Y denotes a single bond, O, NH, CH₂
R denotes H or a C₁-C₈ alkyl, C₃-C₁₄ cycloalkyl, C₆-C₁₀ aryl or C₇-C₁₄ aralkyl group, which may be mono- or polysubstituted by R₅ and whose alkyl-C chain may be interrupted by —O—,
Hal denotes F, Cl, Br, or I
m denotes 0, 1, 2, 3 or 4
and
n denotes 0, 1, 2 or 3,
and pharmaceutically usable derivatives, solvates and stereoisomers thereof, exclusively mixtures thereof in all ratios.

2. Compounds of the formula IA 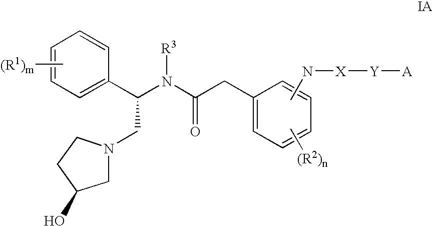
in which R¹, R², R³, X, Y, A, m and n have the meaning indicated in claim 1, and pharmacological usable derivatives, salts, solvates and stereoisomers thereof and mixtures thereof in all ratios.

3. Compound of the formula I and IA, according to claim 1 in which
A denotes phenyl, pyridyl, thienyl or cyclohexyl, each of which is unsubstituted or mono- or polysubstituted by R¹,
R¹ denotes H
R² denotes H or Hal.

4. Medicament of the formula I according to claim 1 in which
A denotes phenyl or naphthyl
and/or
X denotes CO or SO₂
and/or
Y denotes a single bond or NH.

5. Use of the compounds of the formula I and/or IA according to claim 1 and physiologically acceptable salts, solvates and derivatives thereof for the preparation of medicaments for the treatment and/or prophylaxis of irritable bowel syndrome.

6. Pharmaceutical composition, characterised by a content of at least one compound of the formula I and/or IA and/or one of its physiologically acceptable salts, solvates and derivatives according to claim 1 for the treatment and/or prophylaxis of irritable bowel syndrome.

7. Compounds of the formula I according to claim 1 and acceptable salts, solvates and derivatives thereof as medicaments.

8. Use of the compounds of the formula I and/or IA according to claim 1 and physiologically acceptable salts, solvates and derivatives thereof for the preparation of medicaments for the treatment and/or prophylaxis of diseases which can be influenced by kappa agonists.

9. Medicament formulation comprising at least one compound of the formula I and or IA according to claim 1 and/or pharmaceutically usable derivatives, solvates and stereoisomers thereof, including mixtures thereof in all ratios.
